(19) United States
(12) Patent Application Publication
(10) Pub. No.: US 2023/0114666 A1
(43) Pub. Date: Apr. 13, 2023

(54) REAL-TIME SYSTEM FOR MONITORING AND ANALYZING VOLUME OF SUBSTANCE INTRODUCED INTO THE BODY AND VOLUME OF METABOLIC WASTE EXPELLED FROM THE BODY

(71) Applicants: Chih-Wei Chen, New Taipei City (TW); You-Chia Chu, New Taipei City (TW); Zoe Lai, New Taipei City (TW)

(72) Inventors: Chih-Wei Chen, New Taipei City (TW); You-Chia Chu, New Taipei City (TW); Zoe Lai, New Taipei City (TW)

(21) Appl. No.: 17/709,838

(22) Filed: Mar. 31, 2022

(30) Foreign Application Priority Data
Oct. 8, 2021 (TW) 110211857

(51) Int. Cl. G16H 40/67; G16H 80/00; A61B 5/107; A61B 5/00

(52) U.S. Cl. CPC G16H 40/67; G16H 80/00; A61B 5/1073; A61B 5/4848

(57) ABSTRACT

A real-time system for monitoring and analysis includes input and output measurement devices, a cloud medical system, and at least one intelligent device. The input measurement device includes a first microprocessor and a first measurement unit configured to measure a volume of substance introduction, the first microprocessor having a first wireless module for transmitting a signal of the measured substance introduction to the cloud medical system. The output measurement device includes a second microprocessor and a second measurement unit configured to measure a volume of metabolic waste expelled from the body, the second microprocessor having a second wireless module for transmitting a signal of the measured urine to the cloud medical system. A signal of doctor's orders is transmitted from a doctor's orders system to the intelligent device.

### FIELD OF THE INVENTION

The invention relates to real-time systems for monitoring and analysis and more particularly to a real-time system for monitoring and analyzing volume of substance introduced into the body of a patient and volume of metabolic waste expelled from the body of the patient.

### BACKGROUND OF THE INVENTION

Conventionally, a nurse may regularly record food consumed by a patient, volume of urine released from the patient, feces of the patient, and sweat state of the patient every day. The nurse thus can know the health of the patient. Also, the nurse may increase or decrease volume of substance introduced into the body through an intravenous (IV) line if a need arises. Regarding food energy, for example protein contains 4 kcal/g and fat contains 9 kcal/g, Breathing consumes energy. It is possible to calculate energy consumed by a human being based on the times of breathing per minute.

It is reasonable to evaluate functions of the heart and the kidneys of a patient based on volume of substance introduced into the body of the patient and volume of metabolic waste expelled from the body of the patient. For example, a nurse may use IV therapy to deliver fluids, medications and nutrients directly into a patient's vein and use a tube to collect urine released from the patient for examination.

However, a nurse may forget to check the remaining substance to be introduced into the body due to busyness. Also, the patient may be bothered if the nurse turns on light to check the remaining substance. The nurse may bend the waist to see graduation on the weighting scale to collect urine or collect volume of liquid excreted from the peritoneum. The nurse may pour the collected urine into a measuring cup prior to examination. AH of above jobs done by the nurse is labor intensive and time consuming. Also, the measurement is prone to error. Further, for measuring the volume of the collected urine, the originally closed urine excretion system may be hurt. And in turn, it increases the risk of infecting the urine excretion path. Furthermore, the risk of occupational injury to the nurse is greatly increased.

Thus, the need for improvement still exists.

### SUMMARY OF THE INVENTION

It is therefore one object of the invention to provide a real-time system for monitoring and analysis, comprising an input measurement device, an output measurement device, a cloud medical system, and at least one intelligent device wherein the input measurement device includes a first microprocessor and a first measurement unit electrically connected to the first microprocessor and configured to measure a volume of substance introduced into the body of a patient, the first microprocessor having a first wireless module for transmitting a signal of the measured volume of substance introduced into the body of the patient to the cloud medical system; the output measurement device includes a second microprocessor and a second measurement unit electrically connected to the second microprocessor and configured to measure a volume of metabolic waste expelled from the body of the patient, the second microprocessor having a second wireless module for transmitting a signal of the measured volume of metabolic waste expelled from the body of the patient to the cloud medical system; the cloud medical system is configured to receive the signal of the measured volume of substance introduced into the body of the patient from the first wireless module and receive the signal of the measured volume of metabolic waste expelled from the body of the patient from the second wireless module respectively, the cloud medical system including a doctor's orders system and an automatic notification system connected to the doctor's orders system wherein the doctor's orders system provides a suggestion of quantity adjustment of administered medicine to a physician in real time based on changes of the measured volume of substance introduced into the body of the patient and the measured volume of metabolic waste expelled from the body of the patient; and the at least one intelligent device configured to wirelessly connect to the cloud medical system; wherein a signal of doctor's orders is transmitted from the doctor's orders system to the at least one intelligent device.

The invention has the following advantages and benefits in comparison with the conventional art: since the input measurement device can transmit a signal of the measured volume of substance introduced into the body of a patient to the cloud medical system, and the output measurement device can transmit a signal of the measured volume of metabolic waste expelled from the body of the patient to the cloud medical system respectively, a medical employee may remotely operate an intelligent device to know both the volume of substance introduced into the body of a patient and the volume of metabolic waste expelled from the body of the patient in real time. Manual recording and calculation are not involved. The medical employee can remotely know and monitor conditions of the patient in real time. Thus, the medical employee can precisely determine when to introduce medication to a patient, when to increase or decrease liquid medicine introduced into the body, when to increase or decrease the volume of substance introduced into the body, and when to increase or decrease the flow rate of substance introduced into the body. It is possible to adjust the flow rate of substance introduced into the body or the total volume of substance to be administered. This makes remote diagnosis and remote therapy come true. Both time and times of visiting the patient by the medical employee are greatly decreased. Errors caused by manual calculation are decreased to a minimum.

The above and other objects, features and advantages of the invention will become apparent from the following detailed description taken with the accompanying drawings

### BRIEF DESCRIPTION OF THE DRAWINGS

FIG. 1 is a block diagram of a real-time system for monitoring and analyzing volume of substance introduced into the body of a patient and volume of metabolic waste expelled from the body of the patient according to a first preferred embodiment of the invention;

FIG. 2 is a block diagram of a real-time system for monitoring and analyzing volume of substance introduced into the body of a patient and volume of metabolic waste expelled from the body of the patient according to a second preferred embodiment of the invention;

FIG. 3 schematically depicts the doctor's orders system;

FIG. 4 schematically depicts a first environment incorporating the invention; and

FIG. 5 schematically depicts a second environment incorporating the invention.

### DETAILED DESCRIPTION OF THE INVENTION

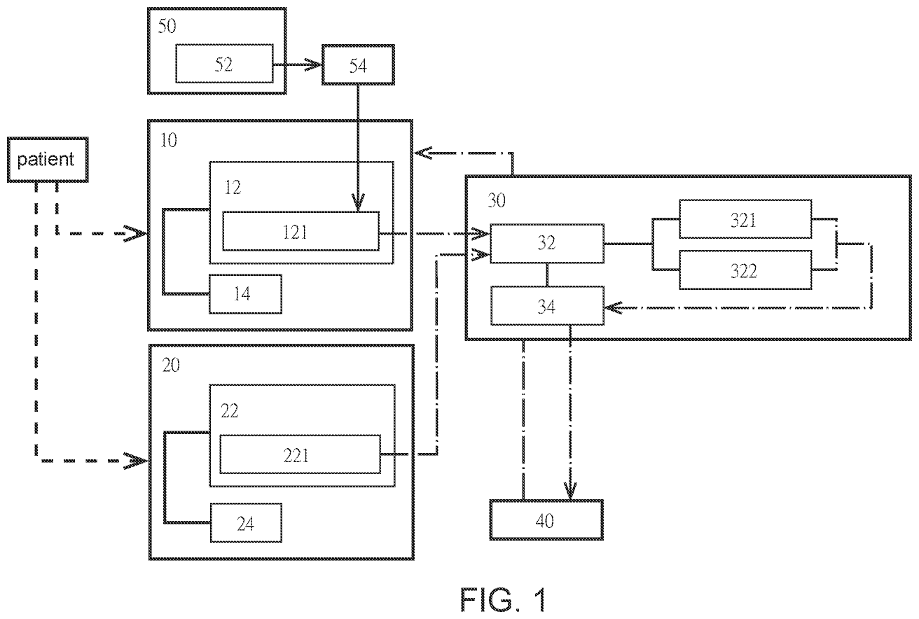

Referring to FIG. 1, a real-time system for monitoring and analyzing volume of substance introduced into the body of a patient and volume of metabolic waste expelled from the body (i.e., urine) of the patient in accordance with a first preferred embodiment of the invention comprises an input measurement device 10, an output measurement device 20, a cloud medical system 30 and at least one intelligent device (one is shown) 40 as discussed in detail below.

The input measurement device 10 includes a first microprocessor 12 and a first measurement unit 14 electrically connected to the first microprocessor 12 and configured to measure a volume of substance introduced into the body of a patient. The first microprocessor 12 includes a first wireless module 121 for transmitting a signal of the measured volume of substance introduced into the body of the patient to the cloud medical system 30. The output measurement device 20 includes a second microprocessor 22 and a second measurement unit 24 electrically connected to the second microprocessor 22 and configured to measure a volume of metabolic waste expelled from the body of the patient. The second microprocessor 22 includes a second wireless module 221 for transmitting a signal of the measured volume of metabolic waste expelled from the body of the patient to the cloud medical system 30. The cloud medical system 30 includes a doctors orders system 32 and an automatic notification system 34. The doctors orders system 32 provides a suggestion of quantity adjustment (i.e., increase or decrease) of administered medicine to a physician in real time based on changes of the measured volume of substance introduced into the body of the patient and the measured volume of metabolic waste expelled from the body of the patient. The intelligent device 40 is wirelessly connected to the cloud medical system 30 so that a signal of doctor's orders can be transmitted from the doctor's orders system 32 to the intelligent device 40. Further, the intelligent device 40 may monitor and notify the patient based on the measured volume of substance introduced into the body of the patient or the measured volume of metabolic waste expelled from the body of the patient.

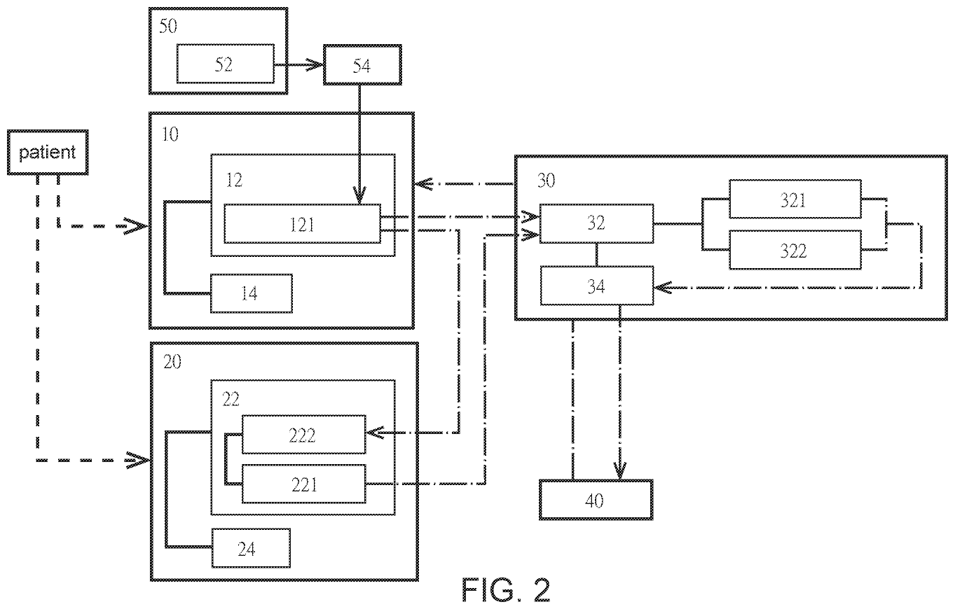

Referring to FIG. 2, a real-time system for monitoring and analyzing volume of substance introduced into the body of a patient and volume of metabolic waste expelled from the body (i.e., urine) of the patient in accordance with a second preferred embodiment of the invention is shown. The characteristics of the second preferred embodiment are substantially the same as that of the first preferred embodiment except the following: the second wireless module 221 can receive a signal of the volume of substance introduced into the body of the patient transmitted from the first wireless module 121. The second microprocessor 22 includes an edge calculation chip 222 for performing edge calculation programs, analyzing changes of the measured volume of substance introduced into the body of the patient and the measured volume of metabolic waste expelled from the body of the patient, and transmitting a signal of the changes to the cloud medical system 30 via the second wireless module 221.

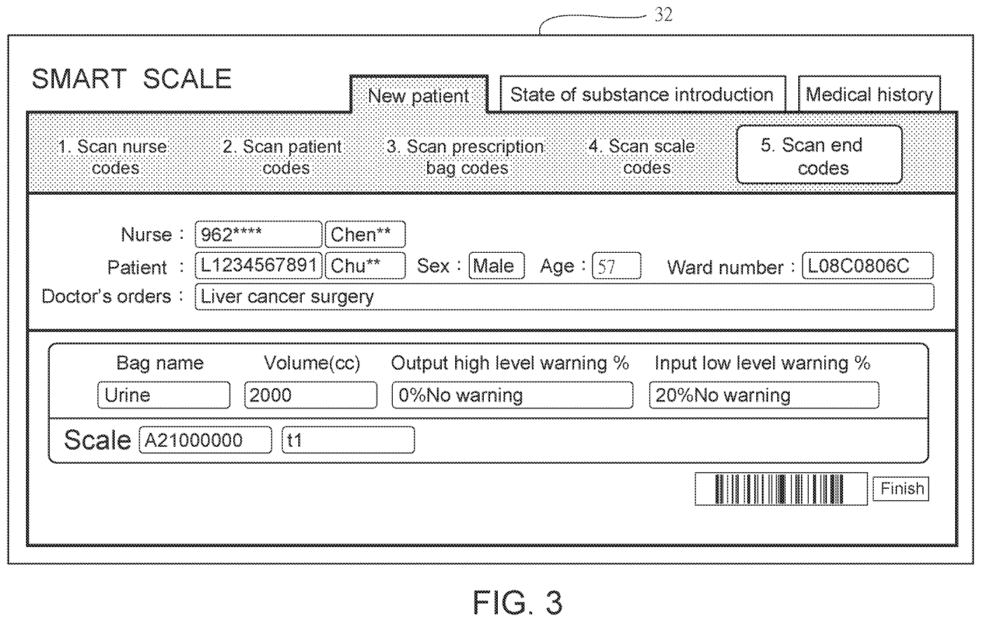

Referring to FIG. 3 in conjunction with FIG. 2, a prescription bag 50 is provided on the input measurement device 10 and includes, printed on its surface, name of drug, quantity of drug, and a barcode 52. A barcode reader 54 can read the printed barcode 52, decode data contained in the barcode 52, and send the data to the first wireless module 121 which in turn transmits a signal of the decoded data to the doctor's orders system 32.

The doctor's orders system 32 is connected to the automatic notification system 34 and includes a high level warning unit 321 and a low level warning unit 322. The high level warning unit 321 can receive a signal transmitted from the second wireless module 221 and the low level warning unit 322 can receive a signal transmitted form the first wireless module 121. An alert value represented in percentage is set by each of the high level warning unit 321 and the low level warning unit 322. For example, a user may manually set or scroll a scrollbar to set a high alert value of 80% for the high level warning unit 321 and set a low alert value of 10% for the low level warning unit 322. In response to the number of pills of the prescription bag 50 decreases to a value less than 10% (i.e., the low alert value), the low level warning unit 322 transmits an alert signal to the automatic notification system 34 which in turn transmits the alert signal to the intelligent device 40. In response to the number of pills of the prescription bag 50 increases to a value more than 80% (i.e., the high alert value), the high level warning unit 321 transmits an alert signal to the automatic notification system 34 which in turn transmits the alert signal to the intelligent device 40.

Preferably, the input measurement device 10 is an intelligent pump for substance introduction, a suspension wireless electronic scale, or a measurement mattress.

Preferably, the output measurement device 20 is a measurement mattress, an intelligent piece of clothing, or a suspension wireless electronic scale.

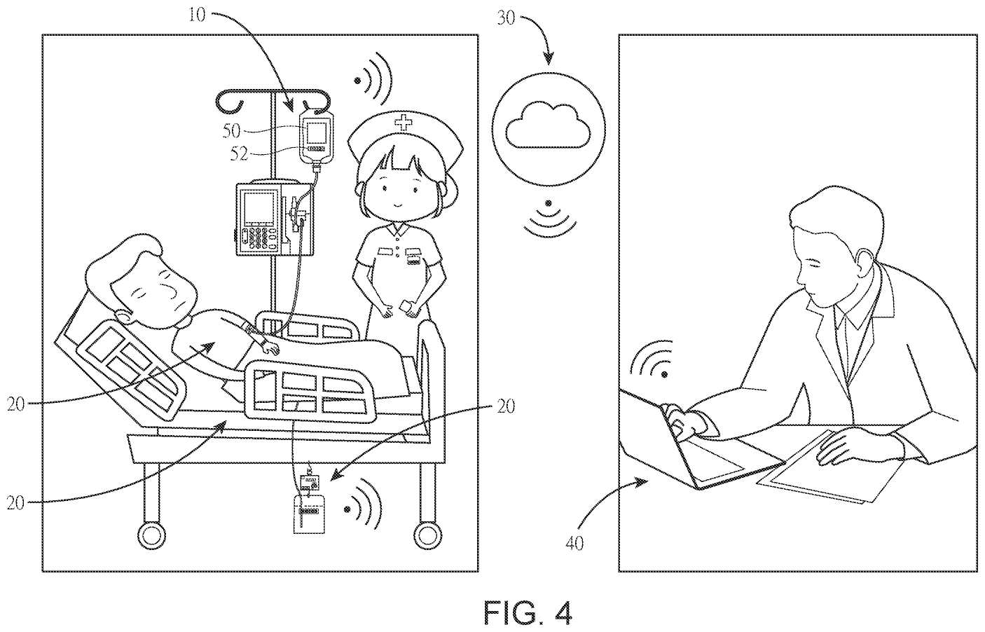

Referring to FIG. 4 in conjunction with FIG. 1, the first measurement unit 14 of the input measurement device 10 may be used to measure volume of substance introduced into the body of a patient. In the embodiment, the input measurement device 10 is, but not limited to an intelligent pump for substance introduction which can transmits signals of both the measured volume of substance introduced into the body and a flow rate of substance introduced into the body to the cloud medical system 30. Alternatively, the input measurement device 10 is a suspension wireless electronic scale or a measurement mattress. For example, a medical employee may use the suspension wireless electronic scale to measure weight of food to be eaten by a patient. And in turn, the suspension wireless electronic scale transmits a signal of the measured weight to the cloud medical system 30. The medical employee may use the measurement mattress to measure weight of the patient and transmit signal of the measured weight to the cloud medical system 30.

The second measurement unit 24 of the output measurement device 20 can measure the volume of metabolic waste expelled from the body of the patient. The output measurement device 20 is implemented as a measurement mattress, an intelligent piece of clothing, or a suspension wireless electronic scale. The measurement mattress can measure a change of weight of the patient in real time. The intelligent piece of clothing can monitor physiological and sweat states of the patient. The suspension wireless electronic scale can measure volume of urine expelled from the patient. Alternatively, the suspension wireless electronic scale can measure volume of fluid expelled from a catheter connected to the stomach, the brain, the chest or the abdomen of the patient. A signal of the measured volume of fluid is sent to the second wireless module 221 which in turn transmits the received signal to the cloud medical system 30.

A physician may use the intelligent device 40 to access a medical record of a patient over the cloud medical system 30. The physician can determine whether to adjust the total volume of substance to be administered based on the calculated net weight in real time. If the total volume of substance adjustment is required, the physician may use the intelligent device 40 to amend the total volume of substance to be administered or the flow rate of the input measurement device 10 through the doctor's orders system 32. Also, data such as the total volume of substance to be administered or the flow rate of substance introduced into the body stored in the doctor's orders system 32 is updated over a wireless intranet of the hospital. The updated data is downloaded to the intelligent pump for substance introduction. Next, a medical employee checks the data and presses a “confirm” key of the intelligent pump for substance introduction if the data is correct. Alternatively, the medical employee many amend the flow rate of substance introduced into the body or the total volume of substance to be administered set by the intelligent pump for substance introduction based on data sent from the doctor's orders system 32. Next, the medical employee presses the “confirm” key of the intelligent pump for substance introduction and verify data stored in the doctor's orders system 32. As an end, the purpose of adjusting the flow rate of substance introduced into the body or the total volume of substance to be administered is achieved. Preferably, the intelligent device 40 is a computer in an office of the hospital, a smartphone, a computer on a medical cart, or a white board in a nurse office.

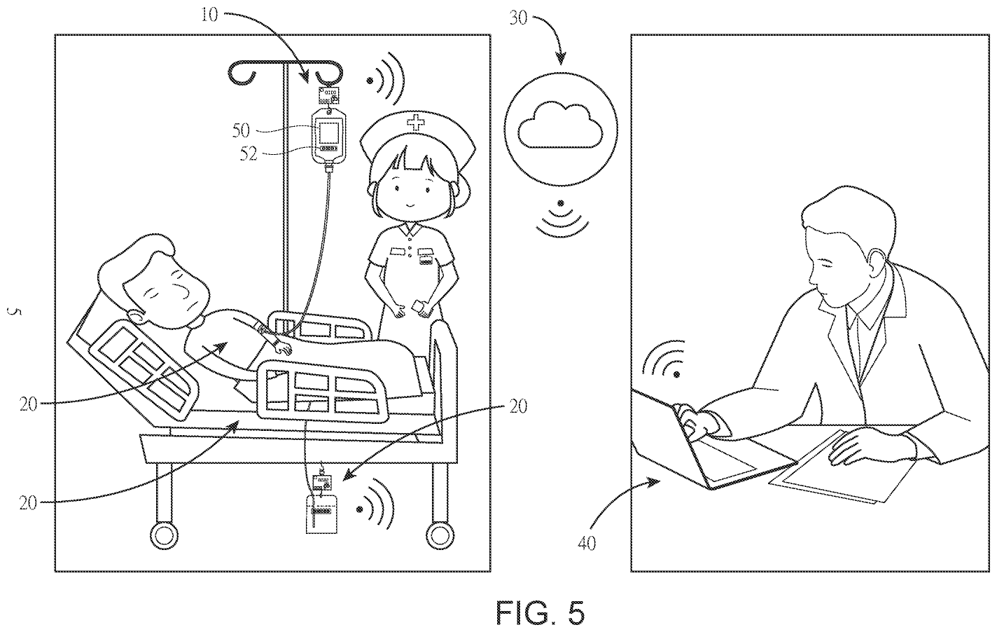

Referring to FIG. 5, it shows that the input measurement device 10 is a suspension wireless electronic scale. The suspension wireless electronic scale transmits a signal of volume of fluid administered to the patient to the cloud medical system 30 in real time.

Following is envisaged by the invention. A medical employee may remotely operate the intelligent device 40 to know both the volume of substance introduced into the body of a patient and the volume of metabolic waste expelled from the body of the patient in real time. Thus, the medical employee can precisely determine when to introduce medication to a patient, when to increase or decrease liquid medicine introduced into the body, when to increase or decrease the volume of substance introduced into the body, and when to increase or decrease the flow rate of substance introduced into the body. In other words, it is possible to adjust the flow rate of substance introduced into the body or the total volume of substance to be administered set by the doctor's orders system 32 through the intelligent device 40. Therefore, the medical employee can remotely know and monitor conditions of a patient in real time. This makes remote diagnosis and remote therapy come true. The medical employee can know conditions of a patient in real time. Finally, the medical employee can know health of the patient by analyzing the obtained data.

While the invention has been described in terms of preferred embodiments, those skilled in the art will recognize that the invention can be practiced with modifications within the spirit and scope of the appended claims.

What is claimed is:

1. A real-time system for monitoring and analysis, comprising an input measurement device, an output measurement device, a cloud medical system, and at least one intelligent device wherein:
the input measurement device includes a first microprocessor and a first measurement unit electrically connected to the first microprocessor and configured to measure a volume of substance introduced into the body of a patient, the first microprocessor having a first wireless module for transmitting a signal of the measured volume of substance introduced into the body of the patient to the cloud medical system;
the output measurement device includes a second microprocessor and a second measurement unit electrically connected to the second microprocessor and configured to measure a volume of metabolic waste expelled from the body of the patient, the second microprocessor having a second wireless module for transmitting a signal of the measured volume of metabolic waste expelled from the body of the patient to the cloud medical system;
the cloud medical system is configured to receive the signal of the measured volume of substance introduced into the body of the patient from the first wireless module and receive the signal of the measured volume of metabolic waste expelled from the body of the patient from the second wireless module respectively, the cloud medical system including a doctor's orders system and an automatic notification system connected to the doctor's orders system wherein the doctor's orders system provides a suggestion of quantity adjustment of administered medicine to a physician in real time based on changes of the measured volume of substance introduced into the body of the patient and the measured volume of metabolic waste expelled from the body of the patient; and
the at least one intelligent device configured to wirelessly connect to the cloud medical system;
wherein a signal of doctor's orders is transmitted from the doctor's orders system to the at least one intelligent device.

2. The real-time system of claim 1, wherein the second wireless module is configured to receive the signal of the volume of substance introduced into the body of the patient transmitted from the first wireless module; and wherein the second microprocessor includes an edge calculation chip for performing edge calculation programs, analyzing changes of the measured volume of substance introduced into the body of the patient and the measured volume of metabolic waste expelled from the body of the patient, and transmitting a signal of the changes to the cloud medical system via the second wireless module.

3. The real-time system of claim 1, wherein the input measurement device is an intelligent pump for substance introduction, a suspension wireless electronic scale, or a measurement mattress.

4. The real-time system of claim 2, wherein the input measurement device is an intelligent pump for substance introduction, a suspension wireless electronic scale, or a measurement mattress.

5. The real-time system of claim 3, further comprising a prescription bag disposed on the input measurement device, the prescription bag including, on its surface, name of drug, quantity of drug, and a barcode; and a barcode reader configured to read the barcode, decode data contained in the barcode, and send the data to the first wireless module which in turn transmits a signal of the decoded data to the doctor's orders system.

6. The real-time system of claim 4, further comprising a prescription bag disposed on the input measurement device, the prescription bag including, on its surface, name of drug, quantity of drug, and a barcode; and a barcode reader configured to read the barcode, decode data contained in the barcode, and send the data to the first wireless module which in turn transmits a signal of the decoded data to the doctor's orders system.

7. The real-time system of claim 5, wherein the doctor's orders system includes a high level warning unit and a low level warning unit, wherein the high level warning unit is configured to receive a signal transmitted from the second wireless module, and wherein the low level warning unit is configured to receive a signal transmitted form the first wireless module, wherein an alert value represented in percentage is set by each of the high level warning unit and the low level warning unit, and wherein in response to a value reaches the alert value set by one of the high level warning unit and the low level warning unit, the high level warning unit or the low level warning unit transmits an alert signal to the automatic notification system which in turn transmits the alert signal to the at least one intelligent device.

8. The real-time system of claim 6, wherein the doctor's orders system includes a high level warning unit and a low level warning unit, wherein the high level warning unit is configured to receive a signal transmitted from the second wireless module, and wherein the low level warning unit is configured to receive a signal transmitted form the first wireless module, wherein an alert value represented in percentage is set by each of the high level warning unit and the low level warning unit, and wherein in response to a value reaches the alert value set by one of the high level warning unit and the low level warning unit, the high level warning unit or the low level warning unit transmits an alert signal to the automatic notification system which in turn transmits the alert signal to the at least one intelligent device.

9. The real-time system of claim 1, wherein the output measurement device is a measurement mattress, an intelligent piece of clothing, or a suspension wireless electronic scale.

10. The real-time system of claim 2, wherein the output measurement device is a measurement mattress, an intelligent piece of clothing, or a suspension wireless electronic scale.
